(19) United States
(12) Patent Application Publication
(10) Pub. No.: US 2015/0343009 A1
(43) Pub. Date: Dec. 3, 2015

(54) ORALLY ADMINISTERED LIQUID FORMULATION

(71) Applicant: TAISHO PHARMACEUTICAL CO., LTD., Tokyo (JP)

(72) Inventors: Takanori IGARASHI, Tokyo (JP); Masaru HATANAKA, Tokyo (JP); Masashi YAMAMOTO, Tokyo (JP); Keisuke NAKAMOTO, Tokyo (JP)

(73) Assignee: TAISHO PHARMACEUTICAL CO., LTD, Tokyo (JP)

(21) Appl. No.: 14/647,881

(22) PCT Filed: Nov. 13, 2013
(86) PCT No.: PCT/JP2013/080662

§ 371 Date: May 28, 2015

(30) Foreign Application Priority Data
Dec. 5, 2012 (JP) 2012-266256

(51) Int. Cl. A61K 36/9068; A61J 1/14; A61K 31/525

(52) U.S. Cl. CPC A61K 36/9068; A61K 31/525; A61J 1/1468

(57) ABSTRACT

An orally administered liquid formulation, characterized by being obtained by blending a) vitamin B2 or an orally administrable sale thereof, b) an herbal-medicine-derived component, c) 1-10 parts by mass of polyvinyl pyrrolidone with respect to 1 part by mass (raw herbal medicine amount) of the herbal-medicine-derived component, and d) 0.02-1.6 parts by mass of at least one species selected from gallic acid and a derivative thereof with respect to 1 part by mass of vitamin B2. This orally administered liquid formulation has the excellent stability of a vitamin B2 species and does not precipitate in a liquid formulation, and can be applied in the fields of medicine and food products.

### TECHNICAL FIELD

The present invention relates to an internal liquid medicine comprising vitamin B2 or salts thereof and a crude drug-derived component. More specifically, the present invention relates to an internal liquid medicine having good stability of vitamin B2s and being free of precipitation in the liquid medicine obtained by simultaneously incorporating polyvinylpyrrolidone and gallic acids into the internal liquid medicine containing vitamin B2 or a salt thereof and a crude drug-derived component, and being applicable to the fields of pharmaceutical drugs and food products.

### BACKGROUND ART

Vitamin B2 and salts thereof are effective in relieving symptoms such as cheilosis angularis, stomatitis, glossitis, cheilitis, conjunctivitis, keratitis, acute and chronic eczema and seborrheic dermatitis, and are also used for supplementation of vitamin B2 at the time of physical fatigue or physical strength decline during pregnancy and breastfeeding periods as well as during and after an illness. Further, since the liquid medicines containing a crude drug extract of Zingiber officinale, Ginseng radix or the like, cause precipitation derived from the crude drug extract during the period of storage, an attempt of using polyvinylpyrrolidone have been made for the purpose of inhibiting such an undesirable precipitation.

Gallic acids are known to have an antioxidative action, and there have so far been disclosed an internal liquid medicine with an improved photostability of thioctic acid and an internal liquid medicine with an inhibited the stability reduction of a triptane compound by the incorporation of gallic acids (Patent Literatures 1 to 3). On the other hand, gallic acids are documented to produce a low water-soluble byproduct by photoirradiation (Non Patent Literature 1), which is believed to cause precipitation.

CITATION LIST

### Patent Literature

Patent Literature 1: JP 2004-305089 A

Patent Literature 2: JP 2012-36166 A

Patent Literature 3: JP 2012-36167 A

### Non Patent Literature

Non Patent Literature 1: J. Phys. Chem. A., 112, 1188-94 (2008)

### SUMMARY OF INVENTION

### Technical Problem

The present inventors have attempted to incorporate polyvinylpyrrolidone into an internal liquid medicine containing vitamin B2 and a crude drug-derived component, but obtained findings that the photostability of vitamin B2s is further reduced.

As a result, an object of the present invention is to provide an internal liquid medicine which inhibits the precipitate production therein and also has a good stability of vitamin B2s.

### Solution to Problem

The present inventors have conducted extensive studies to solve the above problems and found that the inhibition of precipitate production and the improvement of content reduction of vitamin B2s are simultaneously achieved when a specific amount of polyvinylpyrrolidone and a specific amount of gallic acids or derivatives thereof are incorporated, whereby the present invention have been accomplished.

More specifically, the present invention is directed to:

(1) an internal liquid medicine comprising a) vitamin B2 or an internally administrable salt thereof, b) a crude drug-derived component, c) 1 to 10 parts by mass of polyvinylpyrrolidone relative to 1 part by mass (in terms of original crude drug) of the crude drug-derived component, and d) 0.02 to 1.6 parts by mass of at least one species selected from gallic acid and a derivative thereof relative to I part by mass of vitamin B2,
(2) the internal liquid medicine according to (1), wherein the vitamin B2 or an internally administrable salt thereof is selected from riboflavin, riboflavin phosphate, riboflavin butyrate, flavin adenine nucleotide and internally administrable salts thereof,
(3) the internal liquid medicine according to (1), wherein the crude drug-derived component is a Zingiber officinale extract, and
(4) the internal liquid medicine according to any of (1) to (3), which is filled in a container having a light transmittance of 0.1% or more at 449 nm.

### Advantageous Effects of Invention

According to the present invention, an internal liquid medicine can be provided wherein the inhibition of precipitation in the liquid medicine and the improvement of content reduction of vitamin B2 or salts thereof are simultaneously achieved by incorporating both polyvinylpyrrolidone and gallic acids each in specific amounts to the internal liquid medicine containing vitamin B2 or salts thereof and a crude drug-derived component. Further, according to the internal liquid medicine of the present invention, it has become possible to inhibit the content reduction of vitamin B2s and the precipitate production therein even when the medicine is filled in a container having an insufficient light-blocking effect.

### DESCRIPTION OF EMBODIMENTS

Vitamin B2 or internally administrable salts thereof used in the present invention typically refer to those that are edible, and specific examples thereof include riboflavin, riboflavin phosphate, riboflavin butyrate, flavin adenine nucleotide sodium and internally administrable salts thereof. All of them are readily available in the form of commercial products. Examples of the internally administrable salt include salts with inorganic acid such as hydrochloric acid, sulfuric acid, phosphoric acid, nitric acid or hydrobromic acid; salts with organic carboxylic acid such as acetic acid, trifluoroacetic acid, citric acid, benzoic acid, maleic acid, fumaric acid, tartaric acid, succinic acid, tannic acid, butyric acid, enanthic acid, decanoic acid, theoclic acid, salicylic acid, lactic acid, oxalic acid or malic acid; and salts with organic sulfonic acid such as methanesulfonic acid, benzenesulfonic acid or p-toluenesulfonic acid. These salts can be produced by allowing the compound to contact with riboflavin or the like.

Preferably, the amount of vitamin B2 and a salt thereof to be incorporated in the present invention, when converted into riboflavin from the viewpoint of nutritional intake, ranges from 0.01 to 100 mg a day and from 0.00001 to 0.1 parts by mass relative to the total part by mass of the liquid medicine.

Preferably, the crude drug-derived component to be incorporated is in the form of extract. The extract is produced by the conventional method such as a method wherein the extraction from a crude drug raw material is carried out using an extraction solvent at a suitable temperature (a low temperature or with heating). The extraction solvent can be suitably selected depending on a crude drug; however, water, a hydrophilic solvent (ethanol, in particular) or a mixed solvent thereof is preferably used. The crude drug-derived component of the present invention can be used in the form of a liquid extract as it is, an extract diluted with water or the like, a concentrate of liquid extract and a dried product of liquid extract. In other words, the crude drug-derived component of the present invention encompasses all of the dried extracts, soft extracts, fluid extracts, tinctures, crude drug powders and the like. Examples of the usable commercial products include a barrenwort extract, a cistanche salsa extract, a Cuscuta chinensis extract, a Rehmannia glutinosa extract-A, a Poria cocos extract-A, a Polygala tenuifolia extract-A, a Muira Puama extract-A, a Dioscorea batatas liquid extract, an Eucommia ulmoides extract liquid, a Cordyceps sinensis (Berkeley) Saccardo liquid extract, a Zingiber officinale extract, a Zingiber officinale tincture, a cinnamon liquid extract, an Angelica sinensis liquid extract, a Schisandra chinensis liquid extract, an Astragalus membranaceus liquid extract, a Agkistrodon liquid extract-C and a Panax ginseng liquid extract. The amount of crude drug-derived component (in terms of original crude drug) to be incorporated into the present invention is preferably 0.05 to 2 parts by mass relative to the total part by mass of the liquid medicine. Also, the crude drug-derived component used in the present invention is preferably a Zingiber officinale extract.

The polyvinylpyrrolidone used in the present invention refers to those commonly used in food products and pharmaceutical products and specifically includes those having a K value of 17 to 90. All of them are readily available in the form of commercial products. The amount of polyvinylpyrrolidone to be incorporated in the present invention is 1 to 10 parts by mass relative to 1 part by mass (in terms of original crude drug) of the crude drug-derived component. With an amount lower than 1 part by mass, the precipitation inhibitory effect of a preparation may be insufficient, whereas with an amount exceeding 10 parts by mass, the taste derived from polyvinylpyrrolidone makes it difficult to take the medicine.

The gallic acid and derivatives thereof used in the present invention typically refer to those that are edible, and specific examples thereof include gallic acid, methyl gallate, ethyl gallate, propyl gallate, butyl gallate and internally administrable salts thereof. All of them are readily available in the form of commercial products. Examples of the internally administrable salt include ammonium salt, salts with an alkali metal such as sodium and potassium, salts with an alkaline earth metal such as calcium and magnesium, aluminum salts, zinc salts, salts with an organic amine such as triethylamine, ethanolamine, morpholine, pyrrolidine, piperidine, piperazine and dicyclohexylamine, and salts with basic amino acid such as arginine and lysine. These salts can be produced by allowing the compound to contact with gallic acid or the like. The amount of gallic acid and a derivative thereof to be incorporated in the present invention ranges, from the viewpoint of assuring the photostability of vitamin B2, from 0.02 to 1.6 parts by mass, preferably 0.05 to 1.0 part by mass, more preferably 0.1 to 1.0 part by mass, relative to 1 part by mass of vitamin B2. Incorporation thereof in an amount higher than 0.001 parts by mass relative to the total part by mass of the liquid medicine is not preferable, because a water-insoluble byproduct may be produced.

The container used in the present invention has a light transmittance at 449 m of preferably 0.1% or more, more preferably 0.2% or more. Specific examples thereof include transparent glass bottles and brown or brownish glass bottles. The material of container is not particularly limited and examples thereof include glass or plastics.

The pH of the liquid medicine according to the present invention ranges from 2.0 to 7.0, preferably from 2.5 to 5.5. Where necessary, a pH adjusting agent is incorporated in order to maintain the pH of the liquid medicine of the present invention within the above range. Examples of the pH adjusting agent include organic acids such as citric acid, malic acid, fumaric acid, tartaric acid, lactic acid and succinic acid and salts thereof, inorganic acids such as hydrochloric acid and inorganic bases such as sodium hydroxide.

The internal liquid medicine of the present invention can contain, as other components, other vitamins, minerals, amino acids and salts thereof, other crude drugs or crude drug extracts and caffeine as far as the effect of the present invention is not adversely affected.

Further, as necessary, additives such as a sweetener, an acidifier, a thickening stabilizer, an antioxidant, a colorant, a flavor, a corrigent, a preservative, a seasoning, a bittering agent, an enrichment, a solubilizer and an emulsifier can be suitably added as far as the effect of the present invention is not adversely affected.

The internal liquid medicine of the present invention can be prepared by the conventional method without any particular limitation. Typically, the internal liquid medicine is obtained by weighing each of the components, dissolving them in purified water, subsequently adjusting the pH and adding the remaining purified water to adjust to the predetermined volume, followed by, as necessary, filtration and sterilization.

The internal liquid medicine of the present invention is applicable to, for example, pharmaceutical products such as syrup agents and drink agents, other preparations such as quasi medical drugs, and various beverages such as health beverages.

### EXAMPLES

Hereinafter, the present invention is described in further detail with reference to Examples, Comparative Examples and Test Examples.

### Example 1

16.93 mg of riboflavin phosphate, 100 mg of a Zingiber officinale liquid extract (in terms of original crude drug), 5 mg of propyl gallate, 475 mg of citric acid and 1000 mg of polyvinylpyrrolidone were dissolved in purified water; an additional amount of purified water was added thereto to make the total amount 100 mL; the solution was filled in a brown glass bottle having a light transmittance of 0.3% at 449 nm; and the bottle was capped and heat sterilized at 80° C. for 25 minutes to obtain a bottle of transparent internal liquid medicine.

The preparation in the following Comparative Example 1 and Comparative Example 2 was carried out in the same manner as in Example 1. Each of the formulations is shown in Table 1.

TABLE 1

| Component | | Comparative | Comparative | |
|---|---|---|---|---|
| Name | Unit | Example 1 | Example 2 | Example 1 |
| Riboflavin | mg | 16.93 | 16.93 | 16.93 |
| phosphate | | | | |
| Zingiber officinale | mg | 100 | 100 | 100 |
| liquid extract | | | | |
| (in terms of | | | | |
| original crude | | | | |
| drug) | | | | |
| Propyl gallate | mg | 0 | 0 | 5 |
| Citric acid | mg | 475 | 475 | 475 |
| Polyvinylpyrrolidone | mg | 0 | 1000 | 1000 |
| Purified water | | Quantity | Quantity | Quantity |
| | | sufficient to | sufficient to | sufficient to |
| | | make the | make the | make the |
| | | total volume | total volume | total volume |
| | | of 100 mL | of 100 mL | of 100 mL |
| pH | | 2.9 | 2.9 | 2.9 |

### Test Example 1

### Photostability Test of Riboflavin Phosphate

Using a photostability tester (LTL-400D5, manufactured by Nagano Science Co., Ltd.) and a D65 fluorescent lamp as a light source, the test solutions obtained in Example 1, Comparative Example 1 and Comparative Example 2 were exposed to light at 3000 Lux for 200 hours. A residual ratio of riboflavin phosphate in these test solutions was quantitatively determined by liquid chromatographic method (column: ODS-80TS (Tosoh Corporation), mobile phase: water:acetonitrile:phosphoric acid=880:120:1, flow rate: 1 mL/min, detection wavelength: 280 nm). Table 2 shows the residual ratio (%) of the riboflavin phosphate that was maintained after the exposure to light at 3000 Lux for 200 hours relative to the riboflavin phosphate that was present immediately after the preparation.

TABLE 2

| | Comparative | Comparative | |
|---|---|---|---|
| | Example 1 | Example 2 | Example 1 |
| Residual ratio of riboflavin | 79.4 | 73.6 | 86.7 |
| phosphate (%) | | | |

As clearly seen from Table 2, Example 1 had an improved photostability of riboflavin phosphate as compared with Comparative Example 2. The result revealed that the incorporation of propyl gallate could inhibit effectively the photostability reduction of riboflavin phosphate that would be caused by the incorporation of both a Zingiber officinale liquid extract and polyvinylpyrrolidone.

### Example 2 and Example 3

Transparent internal liquid medicines were prepared in the same manner as in Example 1 for the following Example 2, Example 3 and Comparative Example 3. Each of the formulations is shown in Table 3.

TABLE 3

| | | | | Comparative |
|---|---|---|---|---|
| Component Name | Unit | Example 2 | Example 3 | Example 3 |
| Riboflavin phosphate | mg | 16.93 | 16.93 | 16.93 |
| Zingiber officinale | mg | 100 | 100 | 100 |
| liquid extract | | | | |
| (in terms of | | | | |
| original crude | | | | |
| drug) | | | | |
| Propyl gallate | mg | 5 | 1 | 0.15 |
| Citric acid | mg | 475 | 475 | 475 |
| Polyvinylpyrrolidone | mg | 100 | 100 | 100 |
| Purified water | | Quantity | Quantity | Quantity |
| | | sufficient to | sufficient to | sufficient to |
| | | make the | make the | make the |
| | | total volume | total volume | total volume |
| | | of 100 mL | of 100 mL | of 100 mL |
| pH | | 2.9 | 2.9 | 2.9 |

### Test Example 2

### Photostability Test of Riboflavin Phosphate

Using a photostability tester (LTL-400D5, manufactured by Nagano Science Co., Ltd.) and a D65 fluorescent lamp as a light source, the test solutions obtained in Example 2, Example 3 and Comparative Example 3 were exposed to light at 3000 Lux for 400 hours. A residual ratio of the riboflavin phosphate in these test solutions was quantitatively determined by liquid chromatographic method (column: ODS-80TS (Tosoh Corporation), mobile phase: water:acetonitrile:phosphoric acid =880:120:1, flow rate: 1 mL/min, detection wavelength: 280 nm). Table 4 shows the residual ratio (%) of the riboflavin phosphate that was maintained after the exposure to light at 3000 Lux for 400 hours relative to the riboflavin phosphate that was present immediately after the preparation.

TABLE 4

| | | | Comparative |
|---|---|---|---|
| | Example 2 | Example 3 | Example 3 |
| Residual ratio of riboflavin | 70.9 | 58.5 | 53.9 |
| phosphate (%) | | | |

As clearly seen from Table 4, Example 2 and Example 3 had an improved photostability of riboflavin phosphate as compared with Comparative Example 3. The result revealed that for the first time, the addition of 0.06 parts by mass or more of propyl gallate relative to 1 part by mass of vitamin B2 could inhibit effectively the photostability reduction of riboflavin phosphate that would be caused by the incorporation of both a Zingiber officinale liquid extract and polyvinylpyrrolidone.

### INDUSTRIAL APPLICABILITY

The internal liquid medicine having a good stability of vitamin B2s and being free of precipitation in the liquid medicine obtained by the present invention can be used in pharmaceutical products, food products, health beverages and foods for specified health use.

1. An internal liquid medicine comprising a) vitamin B2 or an internally administrable salt thereof, b) a crude drug-derived component, c) 1 to 10 parts by mass of polyvinylpyrrolidone relative to 1 part by mass (in terms of original crude drug) of the crude drug-derived component, and d) 0.02 to 1.6 parts by mass of at least one species selected from gallic acid and a derivative thereof relative to 1 part by mass of vitamin B2.

2. The internal liquid medicine according to claim 1, wherein the vitamin B2 or an internally administrable salt thereof is selected from riboflavin, riboflavin phosphate, riboflavin butyrate, flavin adenine nucleotide and internally administrable salts thereof.

3. The internal liquid medicine according to claim 1, wherein the crude drug-derived component is a Zingiber officinale extract.

4. The internal liquid medicine according to claim 1, which is filled in a container having a light transmittance of 0.1% or more at 449 nm.

5. The internal liquid medicine according to claim 2, which is filled in a container having a light transmittance of 0.1% or more at 449 nm.

6. The internal liquid medicine according to claim 3, which is filled in a container having a light transmittance of 0.1% or more at 449 nm.
